(19) United States
(12) Patent Application Publication
(10) Pub. No.: US 2024/0423464 A1
(43) Pub. Date: Dec. 26, 2024

(54) PULL TAB INCLUDING AN INTEGRATED SEAL FOR USE WITH AN ENDOSCOPE CLEANING DEVICE

(71) Applicant: Covidien LP, Mansfield, MA (US)

(72) Inventors: Garrett Ebersole, Hamden, CT (US); Jacob C. Baril, Norwalk, CT (US); Matthew A. Dinino, Newington, CT (US); Justin J. Thomas, New Haven, CT (US); Saumya Banerjee, Southington, CT (US); Roy Pilletere, Middletown, CT (US); Nicolette Roy, Windsor Locks, CT (US)

(21) Appl. No.: 18/706,105

(22) PCT Filed: Oct. 31, 2022
(86) PCT No.: PCT/IB2022/060462

§ 371 Date: Apr. 30, 2024

Related U.S. Application Data
(60) Provisional application No. 63/274,134, filed on Nov. 1, 2021.

(51) Int. Cl. A61B 1/12; B08B 1/14

(52) U.S. Cl. CPC A61B 1/122; A61B 1/127; A61B 1/128; B08B 1/143

(57) ABSTRACT

An endoscope cleaning device includes a base, a battery pack, and a pull tab. The base includes a cylindrical body defining a canal. The battery pack includes a battery and a heating mechanism thermally coupled to the cylindrical body. The pull tab includes a body and an annular seal detachably coupled to the body. The annular seal is in registration with the canal of the cylindrical body. The pull tab is partially received within the battery pack to inhibit electrical communication between the battery and the heating mechanism. The body of the pull tab is removably supported with respect to the base and the battery pack such that when the pull tab is pulled by a user, the annular seal detaches from the body of the pull tab and remains with respect to the cylindrical body to form a fluid-tight seal against the endoscope.

### FIELD

The disclosure relates to a minimally invasive surgical instrument and, more particularly, to a pull tab including an integrated seal for use with an endoscope cleaning device.

### BACKGROUND

Minimally invasive surgery eliminates the need to make a large incision in a patient, thereby reducing discomfort, recovery time, and many of the deleterious side effects associated with traditional open surgery. Minimally invasive viewing instruments such as, e.g., laparoscopes and endoscopes, provide viewing of internal tissues and/or organs during the minimally invasive surgery. Laparoscopic surgery involves the placement of a laparoscope in a small incision in the abdominal wall of a patient, to view the surgical site. Endoscopic surgery involves the placement of an endoscope in a naturally occurring orifice, e.g., mouth, nose, anus, urethra, or vagina, to view the surgical site. Other minimally invasive surgical procedures include video assisted thoracic surgery and cardiovascular surgery conducted through small incisions between the ribs. These procedures also utilize scopes to view the surgical site.

A typical minimally invasive viewing instrument, e.g., a laparoscope or an endoscope, includes a housing, an elongated lens shaft extending from one end of the housing, and a lens that is provided in a distal end of the elongated lens shaft. A camera viewfinder extends from the other end of the housing. A camera is connected to the housing and transmits images sighted through the lens to an external monitor on which the images are displayed. During a surgical procedure, the distal end portion of the elongated lens shaft is extended into the patient, while the proximal end portion of the elongated lens shaft, the housing and the camera viewfinder remain outside the patient. In this manner, the laparoscope/endoscope is positioned and adjusted to view particular anatomical structures in the surgical field on the monitor.

During insertion of an endoscope or a laparoscope into the body and during the surgical procedure, debris, e.g., organic matter and/or moisture, may be deposited on the lens of the scope. The buildup of debris and condensation on the lens impairs visualization of the surgical site, and often necessitates cleaning of the lens. An endoscope cleaning device may be utilized to remove debris on the lens and to inhibit fogging of the lens upon the endoscope entering a surgical site.

### SUMMARY

In accordance with the disclosure, an endoscope cleaning device includes a base, a battery pack, and a pull tab. The base includes a cylindrical body defining a canal configured to receive an endoscope therein. The battery pack includes a battery and a heating mechanism thermally coupled to the cylindrical body. The pull tab includes a body and an annular seal detachably coupled to the body. The annular seal is in registration with the canal of the cylindrical body. The pull tab is partially received within the battery pack to inhibit electrical communication between the battery and the heating mechanism. The body of the pull tab is removably supported with respect to the base and the battery pack such that when the pull tab is pulled by a user, the annular seal detaches from the body of the pull tab and remains with respect to the cylindrical body to form a fluid-tight seal against the endoscope received through the annular seal and into the canal of the base.

In an aspect, the body of the pull tab may be formed of an electrically insulating material.

In another aspect, the body of the pull tab may be monolithically formed.

In yet another aspect, the annular seal of the pull tab may be formed of an elastic material.

In still yet another aspect, the annular seal of the pull tab may define an opening dimensioned to receive an endoscope having a diameter of about 10 mm in a sealing relation.

In still yet another aspect, the annular seal of the pull tab may define an opening dimensioned to receive an endoscope having a diameter of about 5 mm in a sealing relation.

In an aspect, the endoscope cleaning device may further include a housing covering the battery pack and the cylindrical body of the base. The housing may define an opening to receive the endoscope therethrough. A portion of the pull tab may extend through the opening of the housing.

In another aspect, the body of the pull tab may include an insulating portion configured to be received in the battery pack to inhibit electrical communication between the battery and the heating mechanism and a sealing portion detachably supporting the annular seal.

In another aspect, the cylindrical body may include a proximal portion including a circular guide defining a break.

In yet another aspect, the annular seal of the pull tab may be dimensioned to be received within the circular guide of the cylindrical body.

In accordance with another aspect of the disclosure, a pull tab for use with an endoscope cleaning device includes a first component, a second component, and a seal. The first component includes an insulating portion, a first seal portion, a first grip portion, and a first neck portion extending between the first grip portion and the first seal portion. The second component includes a second seal portion in registration with the first seal portion, a second grip portion, and a second neck portion extending between the first grip portion and the second seal portion. The seal is detachably disposed on the second seal portion of the second component. The seal is configured to receive an endoscope therethrough and form a fluid-tight seal against the endoscope.

In an aspect, the first grip portion of the first component may partially overlap the second grip portion of the second component.

In another aspect, the first neck portion of the first component may be in a superposed relation with the second neck portion of the second component.

In yet another aspect, the second seal portion may define an opening in registration with the seal.

In still yet another aspect, the opening may have a diameter of about 5 mm.

In still yet another aspect, the seal may define a seal opening having a diameter of about 10 mm.

In accordance with yet another aspect of the disclosure, a surgical kit includes an endoscope having a lens at a distal end portion of the endoscope and an endoscope cleaning device. The endoscope cleaning device includes a base, a battery pack, and a pull tab. The base includes a cylindrical body defining a canal configured to receive the endoscope therein. The battery pack includes a heating mechanism thermally coupled to the canal of the base and a battery configured to supply power to the heating mechanism. The pull tab includes a body having an insulation portion configured to inhibit electrical communication between the battery and the heating mechanism, and an annular seal detachably disposed on the body. The pull tab is removably supported with respect to the base and the battery pack such that when the pull tab is pulled by a user, the annular seal detaches from the body of the pull tab and remains with respect to the cylindrical body to form a fluid-tight seal against the endoscope received through the annular seal.

In an aspect, the body of the pull tab may be monolithically formed as a single construct.

In another aspect, at least the annular seal of the pull tab may be formed of an elastic material.

In yet another aspect, the annular seal may be configured to form a fluid-tight seal against an endoscope having a diameter ranging from about 5 mm to about 10 mm.

### BRIEF DESCRIPTION OF THE DRAWINGS

The above and other aspects and features of this disclosure will become more apparent in view of the following detailed description when taken in conjunction with the accompanying drawings wherein like reference numerals identify similar or identical elements.

FIG. 1 is a perspective view of a surgical kit including an endoscope and an endoscope cleaning device including a pull tab in accordance with the disclosure;

FIG. 2 is a perspective view of the endoscope cleaning device of FIG. 1, illustrating a housing of the endoscope cleaning device in phantom;

FIG. 3 an exploded perspective view of the endoscope cleaning device of FIG. 2 with parts separated;

FIG. 4 is a bottom view of the pull tab of FIG. 1;

FIG. 5 is a perspective view of the pull tab of FIG. 4;

FIG. 6 is an exploded perspective view of the pull tab of FIG. 5 with parts separated;

FIG. 7 is a side cross-sectional view of the endoscope cleaning device of FIG. 1 taken along section line 7-7 of FIG. 1;

FIG. 8 is an enlarged view of the indicated area of detail of FIG. 7;

FIG. 9 is a partial cross-sectional view of the endoscope cleaning device of FIG. 8 with a body of the pull tab removed;

FIG. 10 is a perspective view of the endoscope cleaning device of FIG. 2 with the body part of the pull tab removed;

FIG. 11 is a perspective view of the endoscope cleaning device of FIG. 10 illustrating another aspect of a seal of the pull tab;

FIGS. 12 and 13 are perspective views of the endoscope cleaning device of FIG. 1 utilizing a pull tab in accordance with another aspect of the disclosure;

FIG. 14 is a perspective view of a pull tab in accordance with yet another aspect of the disclosure;

FIG. 15 is an exploded perspective view of the pull tab of FIG. 14 with parts separated;

FIG. 16 is a perspective view of the pull tab of FIG. 14 illustrating separating a first component of the pull tab from a second component and a seal of the pull tab; and

FIG. 17 is a perspective view of the pull tab of FIG. 14 illustrating separating the seal from the first and second components of the pull tab.

### DETAILED DESCRIPTION

The pull tab including an integrated seal disclosed herein is described in detail with reference to the drawings, in which like reference numerals designate identical or corresponding elements in each of the several views. As used herein, the term “distal” refers to the portion that is being described which is farther from a user in a conventional use of the pull tab, while the term “proximal” refers to the portion that is being described which is closer to a user in a conventional use of the pull tab. In addition, the terms parallel and perpendicular are understood to include relative configurations that are substantially parallel and substantially perpendicular up to about + or −10 degrees from true parallel and true perpendicular. Further, to the extent consistent, any or all of the aspects detailed herein may be used in conjunction with any or all of the other aspects detailed herein.

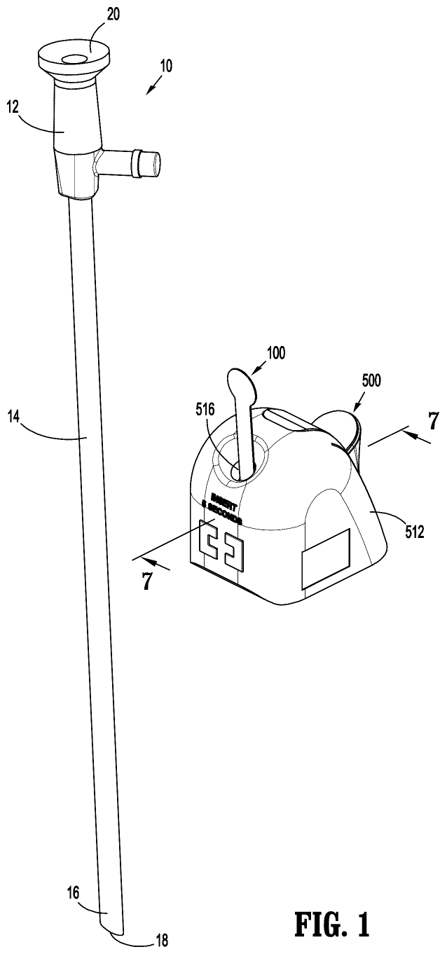

With reference to FIG. 1, an exemplary pull tab including an integrated seal for use with an endoscope cleaning device 500 in accordance with the disclosure is shown generally as a pull tab 100. The endoscope cleaning device 500 is configured to receive an endoscope 10 to clean and warm up a lens 18 at a distal end portion 16 of the endoscope 10 prior to and/or during a surgery. The endoscopic cleaning device 500 reduces fogging of the lens 18 of the endoscope 10 and helps to maintain a good field of vision.

FIG. 1 illustrates the endoscope 10 including a housing 12 and an elongated tubular shaft 14 extending distally from the housing 12 and terminating in the lens 18. The distal end portion 16 of the endoscope 10 includes a number of optical components that produce images of the patient's tissue as known by one skilled in the art. The optical components generally include a window or front element of a lens assembly that is positioned in front of an image sensor or in front of a fiber optic imaging guide that transfers an image to the proximal end of the endoscope 10. Illumination sources such as, e.g., light-emitting diodes, fiber optic or illumination guides, may also be provided. The elongate tubular shaft 14 may be rigid, semi-rigid, or flexible. The housing 12 may include a viewfinder 20 adapted to sight images of a surgical field in the patient, e.g., an abdominal cavity, thoracic cavity, etc., as the position of the endoscope 10 is adjusted to view a particular anatomical structure in the surgical field. A camera is adapted to receive images of the surgical field sighted through the lens 18 and transmit the images to, e.g., an external monitor, on which the images of the surgical field are displayed. That is, a visual display device converts the optical signal into a video signal to produce a video image on the monitor (or for storage on select media). Accordingly, the monitor enables a clinician to view the anatomical structure in the surgical field inside the patient as the surgical procedure is carried out using minimally invasive or endoscopic surgical instruments. Fogging of the lens 18 may occur when the lens 18 is introduced into the surgical site. Further, throughout the surgical procedure, condensation, smoke particles, and biological tissue or matter may contact and build up on the lens 18 of the endoscope 10. This tends to obscure the images of the surgical field as they are displayed on the monitor. To this end, the endoscope cleaning device 500 may be utilized during and/or prior to the surgical procedure to maintain a clear image. In particular, the endoscope cleaning device 500 may be utilized to remove debris such as, e.g., organic matter and/or moisture, from the lens 18 of the endoscope 10 and to reduce fogging of the lens 18 upon the endoscope 10 entering a surgical site.

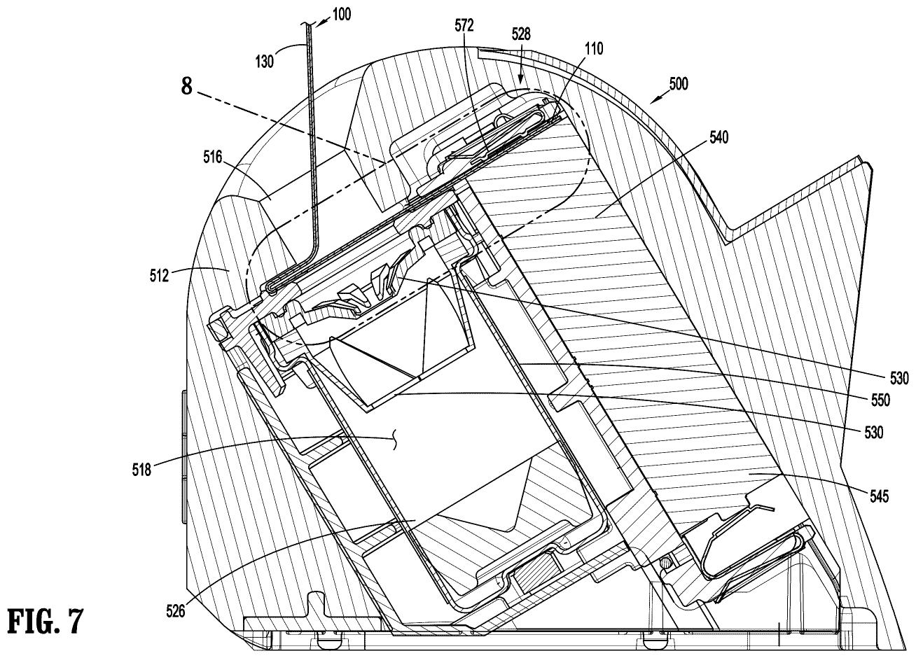

FIGS. 1-3 illustrate the endoscope cleaning device 500 including a housing 512, a base portion 550, a battery pack 540 detachably attached to the base portion 550, and the pull tab 100. The housing 512 is removably coupled to the base portion 550. The housing 512 defines an opening 516. The base portion 550 includes a cylindrical body 555 defining a canal 518 (FIG. 7) for receiving the lens 18 of the endoscope 10. The canal 518 of the base portion 550 is in registration with the opening 516 of the housing 512. The endoscope cleaning device 500 may include a defogging material 526 (FIG. 7) in the canal 518 for treating and inhibiting the lens 18 of the endoscope 10 from fogging during a surgical procedure. The defogging material 526 may include an antifog, lens cleaning agent, or surfactant solution, in the form of a liquid or gel. The endoscope cleaning device 500 may further include a heating mechanism 528 in thermal communication with the canal 518 of the cylindrical body 555 for heating an interior wall of the canal 518 and the defogging material 526 disposed within the canal 518 to further inhibit the lens 18 of the endoscope 10 from fogging. The endoscope cleaning device 500 may further include a seal assembly 530 disposed at least partly within the canal 518. The seal assembly 530 is configured to receive the endoscope 10 therethrough and inhibit the defogging material 526 from spilling out of the canal 518. The seal assembly 530 may be made of a flexible medical grade silicone plastic. In an aspect, a white balancing reference material (not shown) may be disposed within the canal 18 of the cylindrical body 555 to balance the color temperature of the image. The white balancing reference material may be a true white, soft, non-scratch, absorbent material. For example, the white balancing reference material may include a sponge having a white color with a chromaticity of about D-65 or about a D-50 or about D-100. The white balancing reference material may be made out of a low-density foam or other soft material which can be either hydrophobic or hydrophilic. In an aspect, the white balancing reference material is made out of a white medical grade closed cell foam. In an aspect, the housing 512 may include a microfiber fabric on all or part of an outer surface of the housing 512 so that the lens 18 of the endoscope 10 may be wiped thereon and cleaned during a surgical procedure.

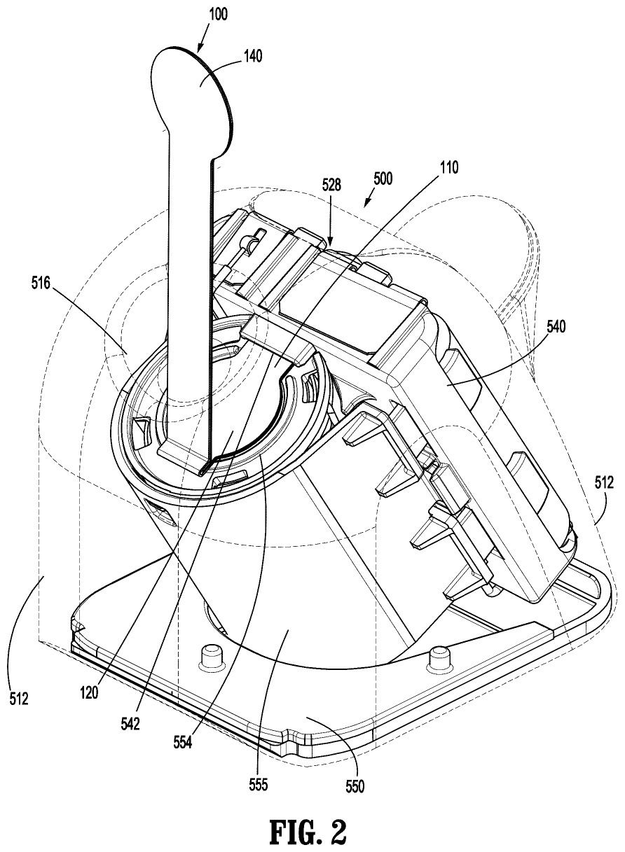

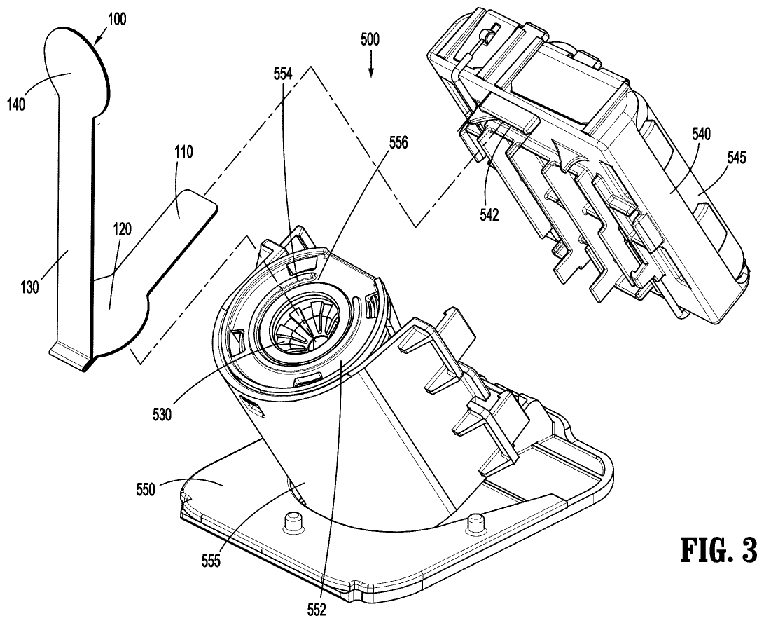

With particular reference to FIGS. 2 and 3, the battery pack 540 is detachably couplable to the base portion 550. The cylindrical body 555 of the base portion 550 defines the canal 518 (FIG. 7) and includes a proximal portion 552 having a circular guide 554 defining a break 556. The circular guide 554 is configured to receive a portion of the pull tab 100 as will be described. The battery pack 540 includes a plurality of batteries 545 and defines a slot 542 configured to slidably receive a portion of the pull tab 100 therein in order to inhibit electrical communication between the batteries 545 and the heating mechanism 528.

The heating mechanism 528 (FIG. 2) may include a heating element such as, e.g., a wound gauge copper wire or nichrome wire, that is connected to the battery pack 540. When activated, i.e., when the pull tab 10 is removed, the battery back 540 energizes the heating element so as to heat the canal 518 and/or the defogging material 526 disposed therein. In an aspect, a thermistor or switch having a thermal component may be placed in the electrical circuit of the heating mechanism 528 to turn off the supply of electricity when a predetermined temperature is reached by the defogging material 526 so as to allow the heating mechanism 528 to maintain a constant temperature of the defogging material 526 above body temperature for an extended period of time while being energized by the battery pack 540.

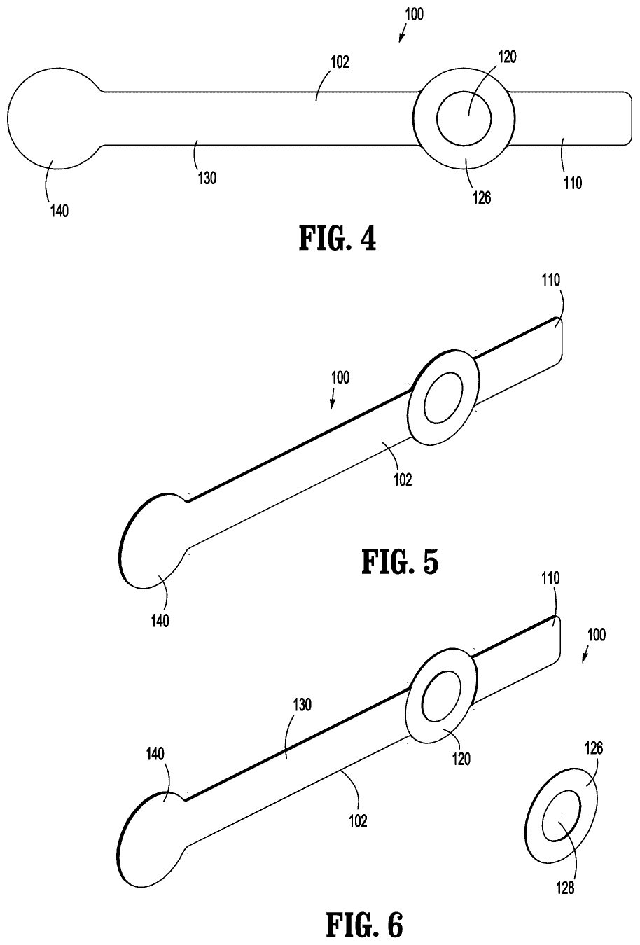

FIGS. 4-6 illustrate the pull tab 100 in accordance with the disclosure. The pull tab 100 includes a body 102 and a seal 126 removably attached to the body 102 with, e.g., an adhesive. The body 102 includes an insulating portion 110, a seal portion 120 supporting the seal 126, a neck portion 130, and a grip portion 140. In an aspect, the body 102 may be monolithically formed. In another aspect, the body 102 may be formed of an electrically insulating material. In yet another aspect, the body 102 may be formed as a single construct. The seal 126 has an annular configuration that defines an opening 128 in registration with the seal assembly 530 (FIG. 3) of the endoscope cleaning device 500. The seal portion 120 is configured to be received within the circular guide 554 (FIG. 3) of the cylindrical body 555 of the base 550 such that the insulating portion 110 of the pull tab 100 extends through the break 556 (FIG. 3) in the circular guide 554 of the base 550 and into the slot 542 (FIG. 3) of the battery back 540, as will be described. A portion of the neck portion 130 of the pull tab 100 is foldable to be secured between the housing 512 (FIG. 1) and the proximal portion 552 (FIG. 3) of the cylindrical body 555 and to direct the grip portion 140 out of the opening 516 (FIG. 1) of the housing 512, as best shown in FIG. 7. Under such a configuration, the grip portion 140 of the pull tab 100 may be pulled by a clinician to remove the insulating portion 110 of the pull tab 100 from the battery pack 540 to energize the heating mechanism 528 (FIG. 2) and to peel or detach the seal 126 of the pull tab 100 from the seal portion 120 of the pull tab 100 in order to place the opening 128 of the seal 126 in registration with the sealing assembly 530 (FIG. 3) disposed in the proximal portion 552 (FIG. 3) of the cylindrical body 555 of the base portion 550. The seal 126 may be formed of, e.g., an elastic, material to form a fluid-tight seal against the endoscope 10 (FIG. 1) received through the opening 128 of the seal 126.

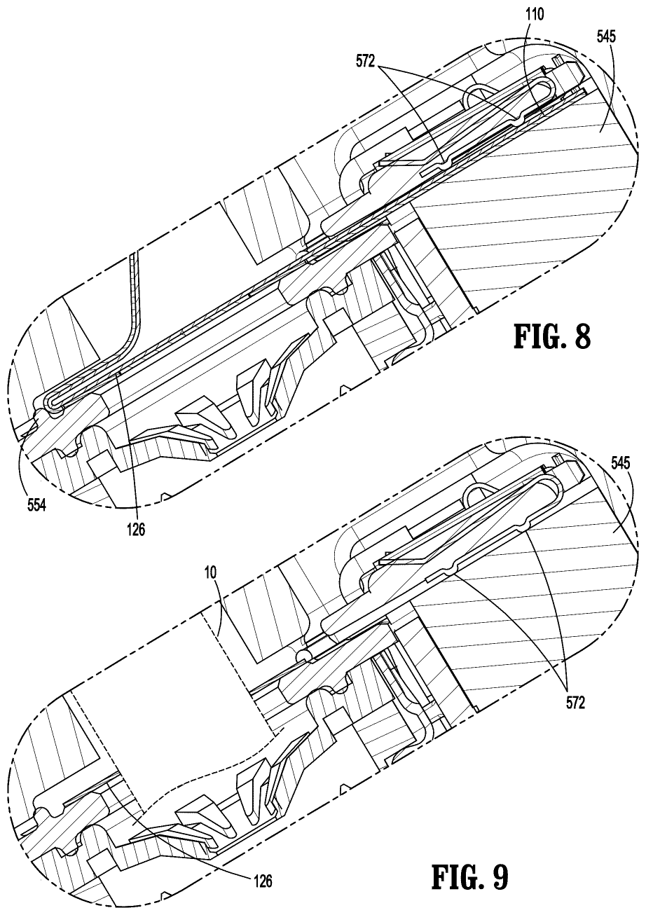

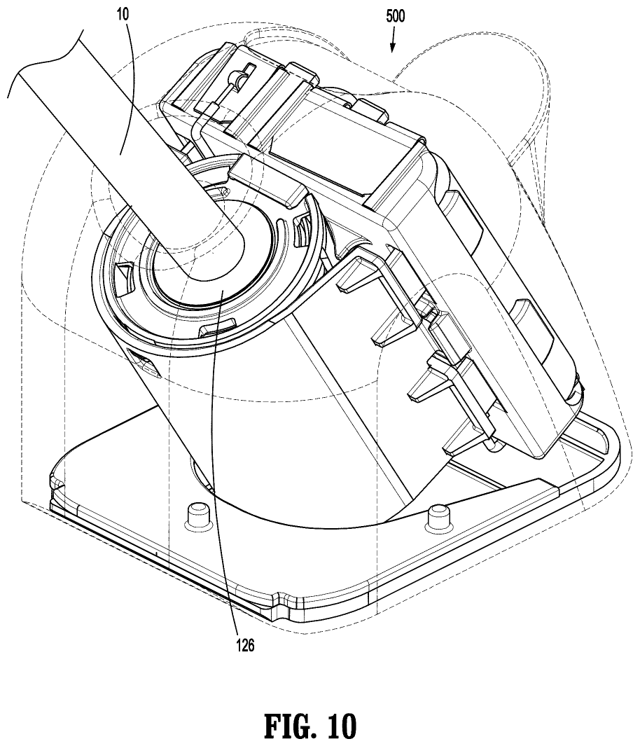

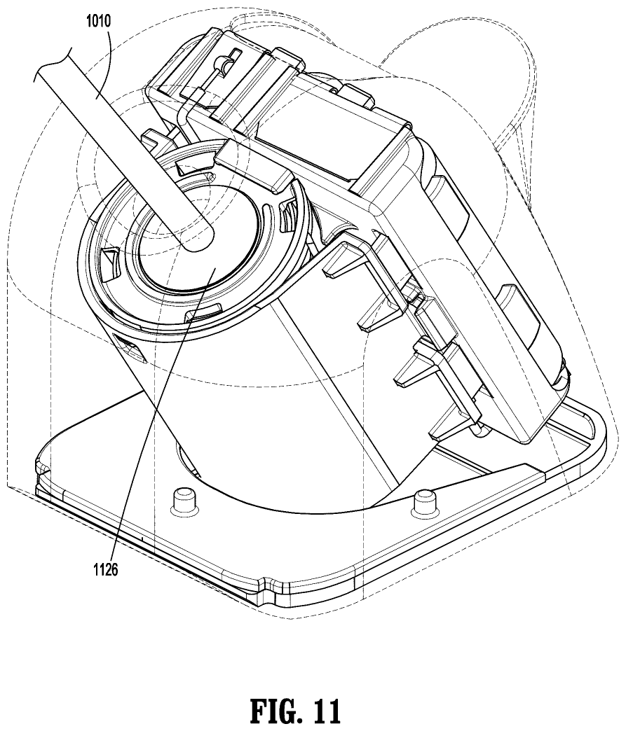

With reference to FIGS. 7 and 8, when the insulating portion 110 of the pull tab 100 is received in the battery pack 540 through the slot 542 (FIG. 3) of the battery pack 540, the insulating portion 110 is interposed between electrical contact portions 572 of the battery pack 540 and the batteries 545 in order to inhibit the batteries 545 from energizing the heating mechanism 528 (FIG. 2). In addition, the seal 126 of the pull tab 100 is detachably supported on the seal portion 120 of the body 102 of the pull tab 100 and is positioned within the circular guide 554 (FIG. 3) of the base portion 550 of the endoscope cleaning device 500. In an aspect, the seal 126 of the pull tab 100 may have a diameter of about 10 mm. The opening 128 of the seal 126 is disposed concentrically with respect to the canal 518 of the cylindrical body 555 of the base portion 550. A portion of the neck portion 130 of the pull tab 100 is folded and secured between the housing 512 of the endoscope cleaning device 500 and the proximal portion 552 of the cylindrical body of the base portion 550. The grip portion 140 of the pull tab extends out of the opening 516 of the housing 512 of the endoscope cleaning device 500. Under such configuration, as the grip portion 140 of the pull tab 100 is pulled away from the housing 512 by the clinician, the portion of the neck portion 130 that is folded and secured between the housing 512 and the proximal portion 552 slides away from the housing 512 and the proximal portion 552 and is removed from the endoscope cleaning device 500 through the opening 516 of the housing 512. At this time, the seal 126 of the pull tab 100 is detached from the seal portion 120 of the body 102 of the pull tab 100 and remains on the proximal portion 552, e.g., within the circular guide 554 of the base portion 550, as shown in FIG. 10. The seal 126 provides a fluid-tight seal against the endoscope 10 extending therethrough and inserted into the canal 518. However, it is contemplated that the pull tab 100 may utilize a smaller seal. For example, in the case of an endoscope 1010 having a smaller diameter than the diameter of the endoscope 10, the pull tab 100 may utilize a seal 1126 having a diameter of about 5 mm, as shown in FIG. 11.

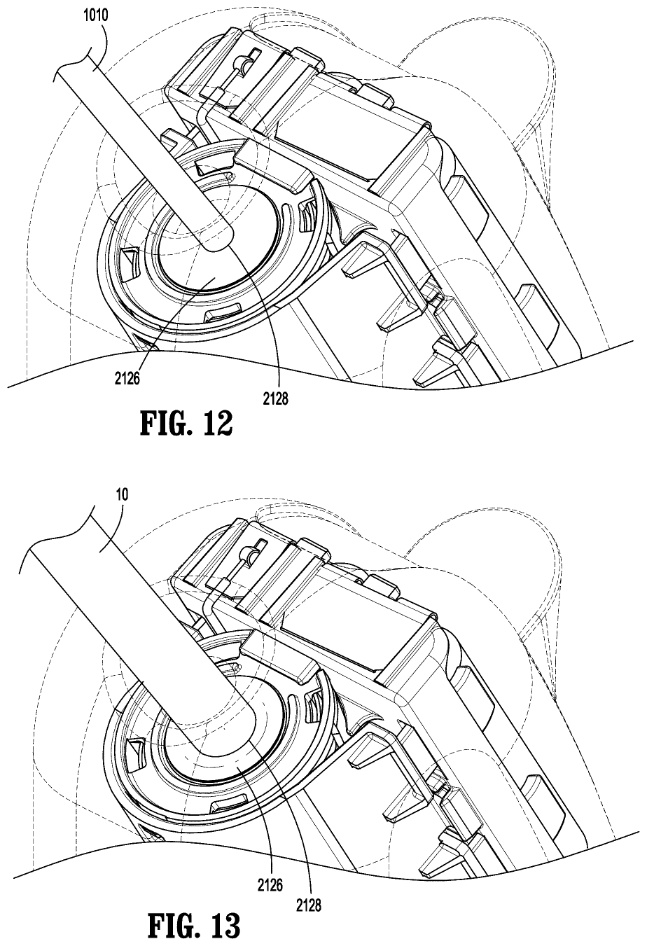

FIGS. 12 and 13 illustrate a seal 2126 formed of an elastic and flexible material such as, e.g., a polymer or gel, to accommodate the endoscopes 10, 1010 of various dimensions. For example, the seal 2126 may define an opening 2128 dimensioned to form a fluid-tight seal against the endoscope 1010 having a diameter of about 5 mm, as shown in FIG. 12. However, when a larger endoscope such as the endoscope 10 having a diameter of, e.g., about 10 mm, is inserted through the seal 2126, the opening 2128 is enlarged while forming a fluid-tight seal against the endoscope 10, as shown in FIG. 13.

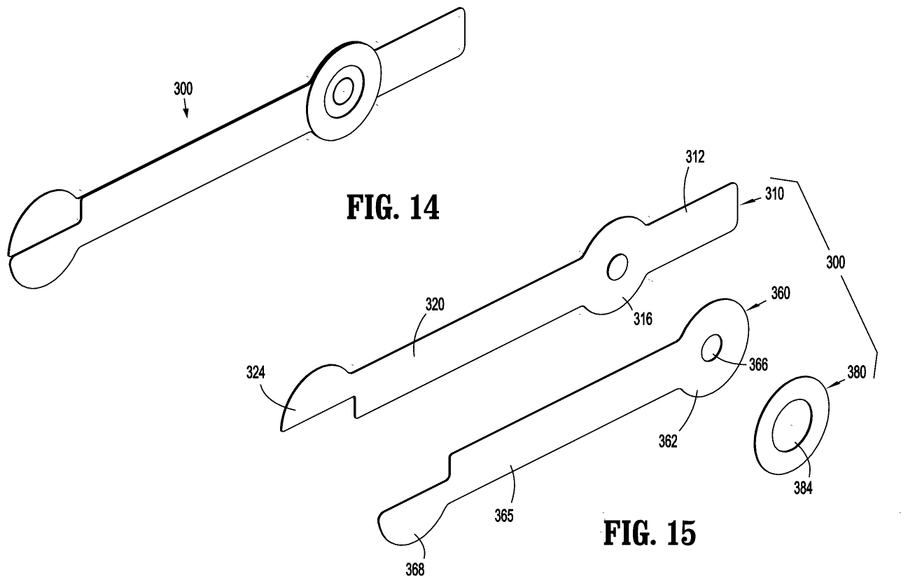

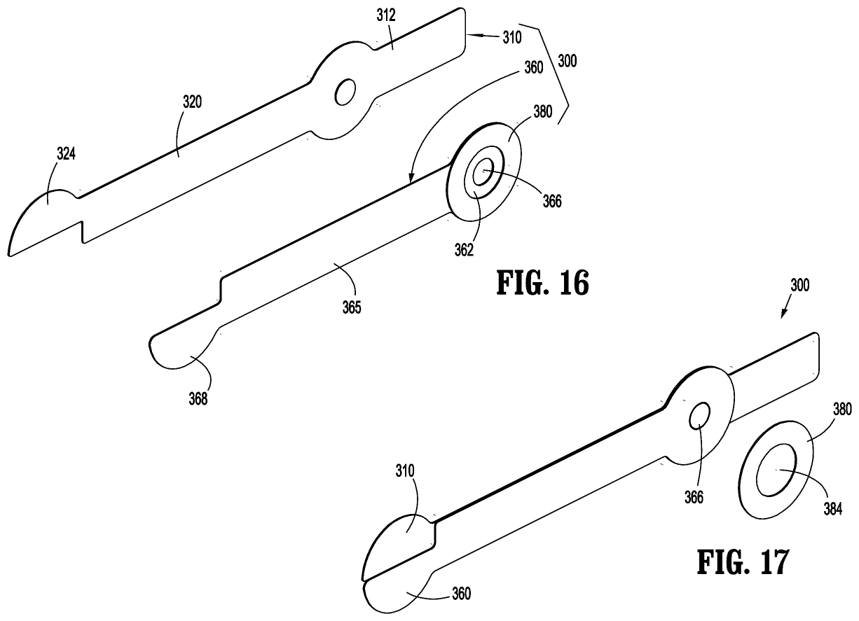

FIGS. 14-17 illustrate a pull tab 300 in accordance with another aspect of the disclosure. The pull tab 300 includes multi-layers that are selectively separable to accommodate various sizes of endoscope. Specifically, the pull tab 300 includes a first component 310, a second component 360, and a seal 380. The first component 310 includes an insulating portion 312, a first seal portion 316, a first neck portion 320, and a first grip portion 324. The insulating portion 312 is configured to be received in the slot 542 (FIG. 3) of the battery pack 540 to be interposed between the batteries 545 (FIG. 8) and the electrical contact portions 572 (FIG. 8) of the battery pack 540. The first seal portion 316 may be in registration with the sealing assembly 530 (FIG. 3) of the base portion 552. The first neck portion 320 extends from the first seal portion 316 and ends in the first grip portion 324. The first grip portion 324 may have, e.g., a semi-circular profile. The second component 360 includes a second seal portion 362, a second neck portion 365, and a second grip portion 368 also having, e.g., a semi-circular profile, that partially overlaps with the first grip portion 324 of the first component 310. The second seal portion 362 of the second component 360 defines an opening 366 having, e.g., a diameter of about 5 mm. The second seal portion 362 is in registration with the first seal portion 316 of the first component 310. The seal 380 is detachably attached to the second seal portion 362 of the second component 360. The seal 380 has an annular configuration defining an opening 384 in communication with the opening 366 of the second component 360. The seal 380 may be attached to the second component 360 with, e.g., an adhesive.

In use, the clinician initially pulls the first component 310 of the pull tab 300 to remove the insulation portion 312 from the slot 542 (FIG. 3) of the battery pack 540 such that the heating mechanism 528 (FIG. 2) is energized by the battery pack 540. After removing the first component 310, the opening 366 of the second component 360 is in communication with the opening 384 of the seal 380. The opening 366 is in registration with the seal assembly 530 (FIG. 3) and the canal 518 (FIG. 7) of the cylindrical body 555 of the base portion 550 (FIG. 3). The opening 366 of the second component 360 provides a diameter of about 5 mm. If the smaller endoscope 1010 is utilized, the second component 360 remains attached to the seal 384 to provide a fluid-tight seal against the endoscope 1010. However, if a larger endoscope, such as the endoscope 10 is utilized, the second component 360 is removed by the clinician by pulling the second grip portion 368 away from the housing 512 (FIG. 1) of the endoscope cleaning device 500. The seal 380 of the pull tab 300 remains on the proximal portion 552 (FIG. 3) of the base portion 550 and provides an opening 384 in registration with the sealing assembly 530 and the canal 518. Under such a configuration, the seal 380 provides a fluid-tight seal against the endoscope 10.

While the disclosure has been shown in the drawings, it is not intended that the disclosure be limited thereto, as it is intended that the disclosure be as broad in scope as the art will allow and that the specification be read likewise. Therefore, the above description should not be construed as limiting, but merely as exemplifications. Those skilled in the art will envision other modifications within the scope and spirit of the claims appended hereto.

What is claimed is:

1. An endoscope cleaning device comprising:
a base including a cylindrical body defining a canal configured to receive an endoscope therein;
a battery pack including a battery and a heating mechanism thermally coupled to the cylindrical body; and
a pull tab including a body and an annular seal detachably coupled to the body, the annular seal in registration with the canal of the cylindrical body, the pull tab partially received within the battery pack to inhibit electrical communication between the battery and the heating mechanism, wherein the body of the pull tab is removably supported with respect to the base and the battery pack such that when the pull tab is pulled by a user, the annular seal detaches from the body of the pull tab and remains with respect to the cylindrical body to form a fluid-tight seal against an endoscope received through the annular seal and into the canal of the base.

2. The endoscope cleaning device according to claim 1, wherein the body of the pull tab is formed of an electrically insulating material.

3. The endoscope cleaning device according to claim 1, wherein the body of the pull tab is monolithically formed.

4. The endoscope cleaning device according to claim 1, wherein the annular seal of the pull tab is formed of an elastic material.

5. The endoscope cleaning device according to claim 1, wherein the annular seal of the pull tab defines an opening dimensioned to receive an endoscope having a diameter of about 10 mm in a sealing relation.

6. The endoscope cleaning device according to claim 1, wherein the annular seal of the pull tab defines an opening dimensioned to receive an endoscope having a diameter of about 5 mm in a sealing relation.

7. The endoscope cleaning device according to claim 1, further including a housing covering the battery pack and the cylindrical body of the base, the housing defining an opening to receive an endoscope therethrough, a portion of the pull tab extending through the opening of the housing.

8. The endoscope cleaning device according to claim 1, wherein the body of the pull tab includes an insulating portion configured to be received in the battery pack to inhibit electrical communication between the battery and the heating mechanism and a sealing portion detachably supporting the annular seal.

9. The endoscope cleaning device according to claim 1, wherein the cylindrical body includes a proximal portion including a circular guide defining a break.

10. The endoscope cleaning device according to claim 9, wherein the annular seal of the pull tab is dimensioned to be received within the circular guide of the cylindrical body.

11. A pull tab for use with an endoscope cleaning device comprising:
a first component including an insulating portion, a first seal portion, a first grip portion, and a first neck portion extending between the first grip portion and the first seal portion;
a second component including a second seal portion in registration with the first seal portion, a second grip portion, and a second neck portion extending between the first grip portion and the second seal portion; and
a seal detachably disposed on the second seal portion of the second component, the seal configured to receive an endoscope therethrough and form a fluid-tight seal against the endoscope.

12. The pull tab according to claim 11, wherein the first grip portion of the first component partially overlaps the second grip portion of the second component.

13. The pull tab according to claim 11, wherein the first neck portion of the first component is in a superposed relation with the second neck portion of the second component.

14. The pull tab according to claim 11, wherein the second seal portion defines an opening in registration with the seal.

15. The pull tab according to claim 14, wherein the opening has a diameter of about 5 mm.

16. The pull tab according to claim 11, wherein the seal defines a seal opening having a diameter of about 10 mm.

17. A surgical kit comprising:
an endoscope having a lens at a distal end portion of the endoscope; and
an endoscope cleaning device including:
a base including a cylindrical body defining a canal configured to receive the endoscope therein;
a battery pack including:
a heating mechanism thermally coupled to the canal of the base; and
a battery configured to supply power to the heating mechanism; and
a pull tab including a body having an insulation portion configured to inhibit electrical communication between the battery and the heating mechanism and an annular seal detachably disposed on the body, wherein the pull tab is removably supported with respect to the base and the battery pack such that when the pull tab is pulled by a user, the annular seal detaches from the body of the pull tab and remains with respect to the cylindrical body to form a fluid-tight seal against the endoscope received through the annular seal.

18. The surgical kit according to claim 17, wherein the body of the pull tab is monolithically formed as a single construct.

19. The surgical kit according to claim 17, wherein at least the annular seal of the pull tab is formed of an elastic material.

20. The surgical kit according to claim 17, wherein the endoscope has a diameter ranging from about 5 mm to about 10 mm and the annular seal is configured to form a fluid-tight seal against the endoscope.
